(19) United States
(12) Patent Application Publication
(10) Pub. No.: US 2022/0110850 A1
(43) Pub. Date: Apr. 14, 2022

(54) LECITHIN-CONTAINING COSMETIC BASE MATERIAL AND COSMETIC PREPARATION IN WHICH SAME IS BLENDED

(71) Applicant: Shiseido Company, Ltd., Chuo-ku, Tokyo (JP)

(72) Inventor: Saori IETANI, Tokyo (JP)

(73) Assignee: Shiseido Company, Ltd., Chuo-ku, Tokyo (JP)

(21) Appl. No.: 17/423,411

(22) PCT Filed: Jan. 7, 2020
(86) PCT No.: PCT/JP2020/000095

§ 371 Date: Jul. 15, 2021

(30) Foreign Application Priority Data
Jan. 16, 2019 (JP) 2019-005307

(51) Int. Cl. A61K 8/55; A61K 8/14; A61K 8/63; A61K 8/34; A61K 8/06; A61Q 19/02; A61Q 19/00

(52) U.S. Cl. CPC A61K 8/553; A61K 8/14; A61K 8/63; A61K 2800/48; A61K 8/068; A61Q 19/02; A61Q 19/007; A61K 8/345

(57) ABSTRACT

The purpose of the present invention is to provide: a cosmetic base material which is capable of stably retaining lecithin in the form of a liposome; and a cosmetic preparation which contains this base material and has excellent usability (such as soft texture when applied to the skin and quick adhesion to the skin) and excellent stability. The present invention relates to: a cosmetic base material which is characterized by containing (a) a liposome that contains (a-1) lecithin and (a-2) an oryzanol, and (b) an aqueous medium that contains (b-1) water and (b-2) a polyhydric alcohol, and which is also characterized in that the ratio of the blending amount of the liposome (a) to the blending amount of the aqueous medium (b), namely (a):(b) is within the range of from 1:99 to 1:32; and a cosmetic preparation which contains a microemulsion part and a liposome part that is composed of this base material.

### TECHNICAL FIELD

The present invention relates to a cosmetic base material containing lecithin. More specifically, the present invention relates to a cosmetic base material which stably contains lecithin in the form of liposome, and further relates to a cosmetic preparation (composition) which comprises a cosmetic base material and has excellent texture such as quick compatibility (fitting) with the skin, absence of stickiness, quick penetration to the skin and soft feeling of the skin after application.

### BACKGROUND ART

Lecithin is known to be excellent in water retention effects and have actions to maintain healthy skin and ameliorate rough skin (for example, Patent Document 1). A phospholipid, the main component of lecithin, is a constituent of the biological membrane and a very safe substance having good affinity to the skin. However, lecithin, when added a low viscous cosmetic preparation such as a skin lotion, is likely to precipitate, and thus various attempts to stably disperse lecithin have been made.

Patent Document 2 discloses a transparent to translucent cosmetic preparation including (a) hydrogenated phospholipid having a phosphatidylcholine content of 60 mass % or more, (b) one or two or more selected from branched higher fatty acids and higher alcohols, (c) a polyhydric alcohol, and (d) water, wherein (a) component:(b) component=1:0.01 to 1:0.4 (mass ratio), and such a cosmetic preparation is considered excellent in the stability and actual feels of efficacy of lecithin (penetration feeling, absence of stickiness, soft skin after application, and the like).

On the other hand, there is an invention focusing on the function of lecithin as an amphiphilic substance, but it is difficult for lecithin alone to maintain the stability of a transparent emulsion, and the stickiness caused by a surfactant which is added to maintain the emulsion stability was problematic (Patent Document 3).

Patent Document 4 discloses a γ-oryzanol-phospholipid complex obtained by removing an organic solvent from a solution in which poorly water-soluble γ-oryzanol and phospholipid (hydrogenated soybean lecithin) are homogeneously dissolved in the organic solvent in advance to cause precipitation and discloses that a transparent emulsified or solubilized composition having high dispersion stability can be obtained when such a complex is dispersed in a water-based formulation. However, the stability and usability of lecithin in this conventional technical system were not considered sufficient.

### CITATION LIST

### Patent Document

### Patent Document 1: JP-A H03-106806

### Patent Document 2: JP-B 5348784

### Patent Document 3: JP-A H02-78432

### Patent Document 4: JP-A 2018-87148

### DISCLOSURE OF INVENTION

### Technical Problem

The prevent invention has an object to obtain a water-based low viscous cosmetic base material capable of further stably containing and retaining lecithin and further provide a cosmetic preparation which stably contains lecithin and excellent in usability (softness, quick compatibility and the like when applied to the skin).

### Solution to Problem

As a result of extensive studies, the present inventor found out that lecithin can be stably retained without precipitating for an extended period of time when a liposome including a complex of lecithin and γ-oryzanol is dispersed in an aqueous medium including a polyhydric alcohol, and the content ratio of the complex and the aqueous medium is adjusted within a predetermined range, whereby the present invention was accomplished. Further, a mode diameter of the liposome including the complex adjusted to about 100 nm or less further enhances the stability.

That is, the present invention provides a cosmetic base material comprising a liposome (a) including (a-1) lecithin and (a-2) oryzanol, and an aqueous medium (b) including (b-1) water and (b-2) a polyhydric alcohol, wherein a ratio of a content of the (a) liposome to a content of the (b) aqueous medium ((a):(b)) ranges from 1:99 to 1:32.

The mode diameter of the (a) liposome in the cosmetic base material of the present invention is preferably about 100 nm or less.

Further, the present invention provides a cosmetic preparation, containing the cosmetic base material which stably contains hydrogenated lecithin in the form of liposome.

### Advantageous Effects of Invention

The cosmetic base material of the present invention can stably retain lecithin, which has been difficult to be stably retained, in the form of liposome. Thus, the cosmetic preparation in which such a cosmetic base material is contained, particularly the cosmetic preparation in combination with a microemulsion, will be excellent in usability (softness, quick compatibility and the like when applied to the skin) in addition to the excellent stability.

### DESCRIPTION OF EMBODIMENTS

The cosmetic base material according to the present invention (hereinafter, also simply referred to as “base material”) includes (a) a liposome and (b) an aqueous medium.

### (a) Liposome

(a) The liposome in the present invention is a vesicle including (a-1) lecithin and (a-2) oryzanol.

(a-1) Lecithin

(a-1) Lecithin (also referred to as phosphatidylcholine) is a type of glycerophospholipid among the phospholipids and the principal constituent of the biological membrane. As industrially available lecithins, soybean-derived soybean lecithin, egg yolk-derived egg yolk lecithin and the like are known. There are also lecithins with various modifications, and in the present invention, hydrogenated lecithin treated by hydrogen addition is preferably used, but not limited thereto.

(a-2) Oryzanol

(a-2) Oryzanol is a component contained in a rice bran oil and an ester of ferulic acid and terpene alcohol. γ-Oryzanol can be added to a cosmetic preparation as a skin conditioning agent or the like but is known to be poorly soluble in ethanol and water and difficult to be stably contained in an aqueous cosmetic preparation.

The (a) liposome in the present invention is preferably prepared using a complex obtained by homogeneously dissolving hydrogenated lecithin as the (a-1) lecithin and γ-oryzanol as the (a-2) oryzanol in an organic solvent and removing the organic solvent from such a solution to simultaneously precipitate the hydrogenated lecithin and the γ-oryzanol.

The thus prepared complex of the hydrogenated lecithin and the γ-oryzanol is preferably adjusted in such a way that a content of the hydrogenated lecithin is 60 to 80 mass %, and a content of the γ-oryzanol is 20 to 40 mass %. A commercial product can be used as such a complex, and preferred examples include “Phytopresome OR” (product name) manufactured by NIPPON FINE CHEMICAL CO., LTD.

The content of the (a) liposome (preferably a complex of hydrogenated lecithin and γ-oryzanol) in the base material of the present invention is 0.0001 to 5 mass %, and preferably 0.01 to 3 mass %.

The base material of the preset invention is obtained by dispersing the (a) liposome in (b) the aqueous medium, and the (b) aqueous medium includes (b-1) water and (b-2) a polyhydric alcohol.

(b-1) Water

The (b-1) water is not particularly limited as long as it can be added to a cosmetic preparation and can be purified water, ion exchanged water, or hot spring water.

(b-2) Polyhydric Alcohol

The (b-2) polyhydric alcohol is not particularly limited and examples include ethylene glycol, diethylene glycol, dipropylene glycol, polyethylene glycol, polypropylene glycol, 1,3-butylene glycol, glycerin, and polyglycerin. As the polyhydric alcohol in the base material of the present invention, 1,3-butylene glycol and glycerin can preferably be added in combination from a viewpoint of usability.

In the base material of the present invention, the mode diameter of the (a) liposome is preferably about 100 nm or less, more preferably about 80 nm or less, and further preferably about 60 nm or less. On the other hand, the mode diameter is preferably about 40 nm or more since the stability reduces when it is too small. The mode diameter is the most frequent particle size in a particle size distribution.

When the content of liposome having a particle size of more than 100 nm increases, the particles are united with each other and likely to aggregate, whereby the stability tends to reduce. Thus, the liposome in the present invention preferably has a homogenized particle size and a reduced content of liposome having a particle size of more than 100 nm. For example, in the present invention, 90% size (d90 size) of the (a) liposome is preferably 200 nm or less, more preferably 150 nm or less, and further preferably 100 nm or less.

The method for microminiaturizing and homogenizing liposome particles is not particularly limited and can be carried out by, for example, using high pressure emulsification. High pressure emulsification is a method of emulsification using a homogenizer (for example, Manton-Gaulin, French press, and microfluidizer) under high pressure of 30 MPa·s or more, preferably 50 MPa·s or more, and particularly preferably 80 MPa·s or more. Repeated high pressure emulsification treatment in multiple times facilitates the microminiaturization and homogenization of liposome particles, and hence the high pressure emulsification treatment is preferably carried out about 2 times or more, for example, 3 times, 4 times, or 5 times.

The base material of the present invention features a ratio of a content of the (a) liposome to a content of the (b) aqueous medium (mass ratio) “(a):(b)” ranging from 1:99 to 1:32. This ratio within the above range can adjust a mode diameter of the liposome prepared by high pressure emulsification to be 100 nm or less. When the content of the liposome increases, slight sliminess may be felt when the cosmetic preparation is applied to the skin, and it is thus further preferable that the above ratio be about 1:99 to 1:50. On the other hand, when a content of the aqueous medium is more than 99 times the content of the liposome, it is difficult to prepare the liposome having a homogenized particle size, whereas such a content is less than 32 times, the liposome has reduced stability and may precipitate.

In the (b) aqueous medium, the content ratio of (b-1) water to the (b-2) polyhydric alcohol (mass ratio) “(b-1):(b-2)” preferably ranges from 5:1 to 1:2. Further, the content of the (b-2) polyhydric alcohol is, from a viewpoint of preservative effects, preferably 40 mass % or more, for example, 40 to 60 mass %, and preferably 45 to 55 mass %, based on the total base material.

The base material of the present invention is a base material in which microminiaturized and homogenized liposome particles are dispersed in aqueous medium and has a substantially transparent appearance. The transparency (L value) of the base material of the present invention is preferably 70 or more, more preferably 80 or more, and further preferably 85 or more. The base material having such high transparency enables to prepare a wide variety of cosmetic preparations in combination with various base materials.

The present invention also relates to cosmetic preparations prepared in combination with the cosmetic base material (liposome part) and other base materials.

Other base materials used in the cosmetic preparation of the present invention include emulsified and solubilized base materials and are not limited thereto. The cosmetic preparation of the present invention contains stably retained lecithin as the liposome part and is thus suitable to be a skin care cosmetic preparation effectively utilizing the actions of lecithin.

Hereinafter, described is an example of a cosmetic preparation (hereinafter, also referred to as “the cosmetic preparation of the present invention”) prepared using a microemulsion as the other base material, but other base materials are not limited to the microemulsion.

The cosmetic preparation of the present invention is prepared by mixing (A) a liposome part and (B) a microemulsion part.

The (A) liposome part consists of the cosmetic base material of the present invention described earlier.

The (B) microemulsion part is an oil-in-water type emulsion in which (c) an oil is emulsified in (e) water using (d) a surfactant. The microemulsion as used herein refers to an emulsion having an average particle size of emulsified oil drops of 10 to 500 nm, and preferably 100 nm or less. The transparency of the (B) microemulsion part is not particularly limited and, for example, those having transparency in which an L value ranges from 1 to 99 are usable.

### (c) Oil

The (c) oil used in the present invention is not particularly limited as long as it is an oil component which can typically be added to cosmetic preparations and, for example, includes silicone oils, ester oils, hydrocarbon oils, higher fatty acids, higher alcohols, and fats and oils.

Examples of the silicone oil include linear silicones such as dimethyl polysiloxane, methyl phenyl polysiloxane, and methyl hydrogen polysiloxane; and cyclic silicones such as octamethylcyclotetrasiloxane and decamethylcyclopentasiloxane.

Examples of the ester oil include isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol di-2-ethylhexanoate, dipentaerythritol fatty acid ester, N-alkyl glycol monoisostearate, neopentyl glycol dicaprate, diisostearyl malate, glycerin di-2-heptyl undecanoate, trimethylolpropane tri-2-ethyl hexanoate, trimethylolpropane triisostearate, pentaerythritol tetra-2-ethylhexanoate, glycerin tri-2-ethylhexanoate, trimethylolpropane triisostearate, cetyl 2-ethylhexanoate, 2-ethylhexyl palmitate, glycerin trimyristate, glyceride tri-2-heptylundecanoate, castor oil fatty acid methyl ester, oleyl oleate, acetoglyceride, 2-heptylundecyl palmitate, diisobutyl adipate, 2-octyldodecyl N-lauroyl-L-glutamate, di-2-heptylundecyl adipate, ethyl laurate, di-2-ethylhexyl sebatate, 2-hexyldecyl myristate, 2-hexyldecyl palmitate, 2-hexyldecyl adipate, diisopropyl sebatate, 2-ethylheyl succinate, ethyl acetate, butyl acetate, amyl acetate, and triethyl citrate.

Examples of the hydrocarbon oil include volatile hydrocarbon oils such as isododecane, isohexadecane, and hydrogenated polyisobutene, and non-volatile hydrocarbon oils such as liquid paraffin, ozokerite, squalene, pristane, paraffin, and ceresine.

Examples of the higher fatty acid include lauric acid, myristic acid, palmitic acid, stearic acid, behenic acid, oleic acid, 12-hydroxystearic acid, undecylenic acid, tall oil acid, isostearic acid, linoleic acid, linolenic acid, eicosapentaenoic acid (EPA), and docosahexaenoic acid (DHA).

Examples of the higher alcohol include linear alcohols such as lauryl alcohol, cetyl alcohol, stearyl alcohol, behenyl alcohol, myristyl alcohol, oleyl alcohol, and cetostearyl alcohol; and branched alcohols such as monostearyl glycerin ether (batyl alcohol), 2-decyltetradecynol, lanolin alcohol, cholesterol, phytosterol, hexyldodecanol, isostearyl alcohol, and octyldodecanol.

Examples of the oils and fats include avocado oil, camellia oil, evening primrose oil, turtle oil, macadamia nut oil, corn oil, mink oil, olive oil, rapeseed oil, egg yolk oil, sesame oil, persic oil, wheat germ oil, sasanqua oil, castor oil, flaxseed oil, safflower oil, cottonseed oil, perilla oil, soybean oil, peanut oil, tea seed oil, kaya oil, rice bran oil, Chinese paulownia oil, Japanese paulownia oil, jojoba oil, germ oil, triglycerin, glycerin trioctanoate, and glycerin triisopalmitate.

### (d) Surfactant

The (d) surfactant used in the present invention is not particularly limited but it is preferable to use a nonionic surfactant. Further, it is preferable, from a viewpoint of the usability, that (d-1) a hydrophilic surfactant having an HLB of 10 or more and (d-2) a lipophilic surfactant having an HLB of less than 10 be added in combination.

(d-1) Hydrophilic Surfactant

The (d-1) hydrophilic surfactant is not particularly limited and examples include polyoxyethylene (10) oleyl ether, polyoxyethylene (15) stearyl ether, polyoxyethylene (20) monostearate, polyoxyethylene (30) glyceryl triisostearate, polyoxyethylene (60) hydrogenated castor oil, and polyoxyethylene (20) sorbitan monolaurate.

(d-2) Lipophilic Surfactant

The (d-2) lipophilic surfactant is not particularly limited, and examples include stearyl stearate, diglyceryl distearate, diglyceryl diisostearate (polyglyceryl-2 diisostearate), polyoxyethylene (3) oleyl ether, polyoxyethylene (9) lauryl ether stearate, ethylene glycol monostearate, polyoxyethylene (3) glyceryl trioleate, and polyoxyethylene (4) sorbitan tristearate.

### (e) Water

The (B) microemulsion part in the present invention is an oil-in-water type emulsion in which the (c) oil is emulsified in (e) water using the (d) surfactant. The preparation method is not particularly limited, and a method commonly used for cosmetic preparations can be used.

The content of the (c) oil and the (d) surfactant in the (B) microemulsion part is not particularly limited as long as a stable microemulsion can be prepared by employing such a content. The content of the (c) oil is typically 0.05 to 5 mass %, and preferably 0.5 to 3 mass %. The content of the (d) surfactant is typically 0.1 to 4 mass %, and preferably 0.5 to 2 mass %.

The cosmetic preparation of the present invention can be prepared by mixing the (A) liposome part and the (B) microemulsion part. The content ratio of the (A) liposome part to the (B) microemulsion part is not particularly limited. However, when the content of the liposome is high, sliminess may be felt when the cosmetic preparation is applied to the skin. Thus, the finally obtained cosmetic preparation is prepared in such a way that the content of the (a) liposome (that is, the total content of lecithin and oryzanol) is 0.00001 to 0.2 mass %, preferably 0.001 to 0.15 mass %, and more preferably 0.01 to 0.1 mass %, based on the total amount of the cosmetic preparation.

### (f) Thickener

The (f) thickener, when added to the cosmetic preparation of the present invention, enhances smoothness when the cosmetic preparation is applied to the skin. Examples of the thickener used preferably in the present invention include plant-based polymers such as gum arabic, tragacanth gum, galactan, guar gum, carob gum, gum karaya, gellan gum, and carrageenan; microbial polymers such as xanthan gum, dextran, succinoglycan, and pullulan; animal-based polymers such as collagen, casein, albumin, and gelatin; starch-based polymers such as carboxymethyl starch, and methyl hydroxypropyl starch; cellulose-based polymers such as methyl cellulose, ethyl cellulose, methyl hydroxypropyl cellulose, hydroxy ethyl cellulose, sodium cellulose sulfate, hydroxypropyl cellulose, carboxymethylcellulose sodium, and crystalline cellulose; alginic acid-based polymers such as sodium alginate, and propylene glycol alginate; vinyl-based polymers such as polyvinyl alcohol, polyvinyl acetate, polyvinyl methyl ether, polyvinylpyrrolidone, copolymers of vinylpyrrolidone and vinyl acetate, and carboxy vinyl polymers; and acrylic-based polymers such as sodium polyacrylate, polyethyl acrylate, alkanolamine polyacrylate, copolymers of alkyl methacrylate and dimethylamino ethyl methacrylate, poly 2-acrylamido-2-methylpropanesulfonic acid, and polymethacryloyloxytrimethyl ammonium.

Of the (f) thickeners described above, sodium polyacrylate, when added, provides a notable enhancement effect on smoothness. Sodium polyacrylate preferably usable are those having a weight-average molecular weight of about 100000 to about 10000000. Of these, further preferable are those having a weight-average molecular weight of 500000 to 8000000 and also a content of polymers having a molecular weight of 10000000 or more of 10 mass % or less. Examples of the commercial product of sodium polyacrylate include ARONVIS SX (manufactured by Toagosei Co., Ltd.). The content of sodium polyacrylate is 0.005 to 0.2 mass %, and preferably 0.005 to 0.1 mass %.

### (g) PEG/PPG Dialkyl Ether

(g) PEG/PPG dialkyl ether, when added to the cosmetic preparation of the present invention, can further improve the smoothness. Specific examples of (g) PEG/PPG dialkyl ether include PEG/PPG-10/10 dimethyl ether, PEG/PPG-9/2 dimethyl ether, PEG/PPG-14/7 dimethyl ether, PEG/PPG-6/14 dimethyl ether, PEG/PPG-15/5 dimethyl ether, PEG/PPG-25/25 dimethyl ether, PEG/PPG-10/10 diethyl ether, PEG/PPG-10/10 dipropyl ether, and PEG/PPG-10/10 dibutyl ether. Of these, PEG/PPG-14/7 dimethyl ether is preferably added.

The content of (g) PEG/PPG dialkyl ether is not particularly limited and 0.1 to 5 mass %, and preferably 0.1 to 3 mass %, based on the total amount of the cosmetic preparation.

### (h) Moisturizer

(h) The moisturizer is preferably added to the cosmetic preparation of the present invention to demonstrate the effect to soften the skin in collaboration with lecithin which is stably contained.

Examples of the (h) moisturizer include diglycerin, dipropylene glycol, propanediol, PEG-20, erythritol, and sodium acetylhyaluronate. Of these, diglycerin is preferably used.

The content of the (h) moisturizer is 0.001 to 20 mass %, and preferably 0.001 to 10 mass %.

Other optional components typically used in cosmetic preparations and quasi-drugs can be added to the cosmetic preparation of the present invention in addition to the above components within the range of not affecting the effects of the present invention. The other optional components are not limited, and examples include a dispersant, a preservative, various drugs, a buffer, and a fragrance.

The cosmetic preparation of the present invention is suitable to be a skin care cosmetic preparation in which lecithin is stably contained. The form thereof is not particularly limited, and the cosmetic preparation can be provided in the form of, for example, lotion, milky lotion, skin lotion, and serum.

### EXAMPLES

Hereinafter, the present invention will be further described in detail in reference to Examples but is not at all limited to these descriptions. The content means mass % unless otherwise stated.

### (A) Cosmetic Base Material

A liposome dispersion including a complex of lecithin and oryzanol (cosmetic base material) was prepared by the formulation shown in the following Table 1. High pressure emulsification at 100 Mpa·s (twice) was carried out for the preparation. The liposome particle size distribution of the obtained liposome dispersion was measured.

TABLE 1

| | | |
|---|---|---|
| Example 1 | Example 2 | Example 3 |

(a) Liposome

| | | | |
|---|---|---|---|
| Hydrogenated lecithin- | 1 | 2 | 3 |
| oryzanol complex (*) | | | |

(b) Aqueous medium

| | | | |
|---|---|---|---|
| Ion exchanged water | 81.5 | 63.5 | 45.5 |
| Glycerin | 9 | 18 | 27 |
| 1,3-Butylene glycol | 8 | 16 | 24 |

Preservative

| | | | |
|---|---|---|---|
| Phenoxyethanol | 0.5 | 0.5 | 0.5 |
| (a):(b) | 1:99 | 1:49 | 1:32 |
| pH | 7.05 | 6.9 | 6.95 |
| L VALUE | 79 | 84 | 89 |
| Mode diameter | About 80 nm | About 50 nm | About 20 nm |
| d90 Size | About 200 nm | About 150 nm | About 100 nm |

(*) Phytopresome OR

As shown in Table 1, when the ratio of the blend amount of the liposome structural component (hydrogenated lecithin-oryzanol complex) to the blend amount of the aqueous medium (including water+polyhydric alcohol) ranged from 1:99 to 1:32, liposome dispersions (base material) in which the mode diameter was 100 nm or less and d90 size was 200 nm or less were obtained. In all Examples 1 to 3, lecithin was stably retained without precipitating. However, under the same preparation conditions, as liposome concentrations increased, the microminiaturization and homogenization of liposome particles seemingly tended to be facilitated.

The composition described in the following Table 2 was subjected to high pressure emulsification 5 times to prepare a cosmetic base material (Example 4).

TABLE 2

Example 4

(a) Liposome

| | |
|---|---|
| Hydrogenated lecithin- | 1 |
| oryzanol complex (*) | |

(b) Aqueous medium

| | |
|---|---|
| Ion exchanged water | 75.55 |
| Glycerin | 10 |
| Dipropylene glycol | 5 |
| 1,3-Butylene glycol | 8 |

Preservative

| | |
|---|---|
| Methyl paraben | 0.15 |
| Phenoxyethanol | 0.3 |
| Mode diameter | 50 nm |
| L VALUE | 85 |

(*) Phytopresome OR

Cosmetic preparations were prepared by adding the base material of the above Example 4 as the liposome part (A) in combination with the microemulsion part (B), which was separately prepared by the composition shown in the following Table 3. The obtained cosmetic preparation of each Example was evaluated for appearance, usability and the like in accordance with the following criteria.

A⁺: Extremely excellent

A: Very excellent

B: Excellent

C: Fair

D: Poor

TABLE 3

| | | | |
|---|---|---|---|
| | | Comparative | Comparative |
| Example 5 | Example 6 | Example 1 | Example 2 |

(A) Liposome part

| | | | | |
|---|---|---|---|---|
| Base material | 10 | 10 | — | — |
| of Example 4 | | | | |

(B) Microemulsion part

| | | | | |
|---|---|---|---|---|
| Ion exchanged | Balance | Balance | Balance | Balance |
| water | | | | |
| Ethanol | 7 | 7 | 7 | 7 |
| Glycerin | 7 | — | 7 | 7 |
| Diglycerin | 3 | 3 | 3 | 3 |
| Dipropylene | 4 | 4 | 4 | 4 |
| glycol | | | | |
| 1,3-Butylene | 1 | 1 | 1 | 1 |
| glycol | | | | |
| PEG/PPG-14/7 | 1.5 | 1.5 | 1.5 | 5 |
| Dimethyl ether | | | | |
| Sodium | 0.03 | 0.03 | 0.03 | 0.03 |
| polyacrylate Na | | | | |
| Polyoxyethylene | 1 | 1 | 1 | 1 |
| (60) hydrogenated | | | | |
| castor oil | | | | |
| Polyglyceryl-2 | 0.5 | 0.5 | 0.5 | 0.5 |
| diisostearate | | | | |
| Diphenylsiloxy | 1 | 1 | 1 | 1 |
| phenyl tri- | | | | |
| methicone | | | | |
| Isostearyl | 0.5 | 0.5 | 0.5 | 0.5 |
| alcohol | | | | |
| Dipotassium | 0.05 | 0.05 | 0.05 | 0.05 |
| glycyrrhizinate | | | | |
| Buffer | q.s. | q.s. | q.s. | q.s. |
| Chelating agent | q.s. | q.s. | q.s. | q.s. |
| Phenoxyethanol | q.s. | q.s. | q.s. | q.s. |
| Fragrance | q.s. | q.s. | q.s. | q.s. |
| Total | 100 | 100 | 100 | 100 |
| pH | 6.55 | 6.45 | 6.55 | 6.67 |
| L VALUE | 53 | 42 | 51 | 55 |
| Penetration | A⁺ | A⁺ | B | C |
| feeling | | | | |
| Absence of | B | A | B | C |
| sliminess | | | | |
| Absence of | A | A | D | C |
| friction when | | | | |
| drying | | | | |
| Absence of | A | A | D | C |
| stickiness | | | | |
| Softness when | A⁺ | A⁺ | D | C |
| applied | | | | |

As shown in Table 3, Example 5 in which the base material of the present invention as the liposome part (A) was added in combination with the microemulsion part (B) had the results demonstrating “Excellent” in all of the items evaluated. Example 6 in which glycerin was removed from the microemulsion part (B) of Example 5 was further enhanced in the absence of sliminess. To the contrary, Comparative Example 1 in which the liposome part (A) including lecithin was not added lacked the absence of friction, stickiness and softness when applied. Comparative Example 2 in which the content of PEG/PPG dialkyl ether was increased in the microemulsion part (B) of Comparative Example 1 demonstrated improvement of the absence of friction, stickiness and softness when applied but reduced the penetration feeling and the absence of sliminess.

Hereinafter, other Formulation Examples of the cosmetic preparations of the present invention are presented.

### FORMULATION EXAMPLE 1

### Cosmetic Preparation

| | |
|---|---|
| Components added | content (mass %) |

(A) Liposome part

| | |
|---|---|
| Base material of Example 4 | 10 |

(B) Microemulsion part

| | |
|---|---|
| Glycerin | 8.25 |
| Diglycerin | 3 |
| Dipropylene glycol | 9 |
| 1,3-Butylene glycol | 1 |
| PEG/PPG-14/7 Dimethyl ether | 1.5 |
| Sodium polyacrylate | 0.02 |
| Polyoxyethylene (60) hydrogenated castor oil | 1.0 |
| Polyglyceryl-2 diisostearate | 0.5 |
| Diphenylsiloxy phenyl trimethicone | 1.0 |
| Isostearyl alcohol | 0.5 |
| Dipotassium glycyrrhizinate | 0.05 |
| Buffer | q.s. |
| Chelate agent | q.s. |
| Phenoxyethanol | q.s. |
| Ion exchanged water | balance |
| Total | 100 |

### FORMULATION EXAMPLE 2

### Translucent Skin Lotion

| | |
|---|---|
| Components added | content (mass %) |

(A) Liposome part

| | |
|---|---|
| Base material of Example 4 | 1 |

(B) Microemulsion part

| | |
|---|---|
| Ethanol | 5 |
| Glycerin | 5 |
| Dipropylene glycol | 7 |
| 1,3-Butylene glycol | 5 |
| Erythritol | 1 |
| Xylitol | 1 |
| PEG/PPG-17/4 Dimethyl ether | 3 |
| POE (30) Phytosterol | 0.15 |
| Sorbitan sesquioleate | 0.05 |
| Isostearyl alcohol | 0.5 |
| α-Olefin oligomer | 0.1 |
| Xanthan gum | 0.1 |
| Citric acid | q.s. |
| Sodium citrate | q.s. |
| Sodium metaphosphate | q.s. |
| Phenoxyethanol | q.s. |
| Fragrance | q.s. |
| Ion exchanged water | balance |
| Total | 100 |

### FORMULATION EXAMPLE 3

### Translucent Skin Lotion

| | |
|---|---|
| Components added | content (mass %) |

(A) Liposome part

| | |
|---|---|
| Base material of Example 4 | 1 |

(B) Microemulsion part

| | |
|---|---|
| Glycerin | 10 |
| Dipropylene glycol | 7 |
| 1,3-Butylene glycol | 5 |
| POE (10) methyl glucoside | 2 |
| Erythritol | 1 |
| PEG-400 | 1 |
| PEG/PPG-14/7 Dimethyl ether | 1 |
| Polyoxyethylene (60) hydrogenated castor oil | 0.2 |
| Polyglyceryl-2 diisostearate | 0.1 |
| Isostearyl alcohol | 0.3 |
| Hydroxyethyl cellulose | 0.1 |
| Citric acid | q.s. |
| Sodium citrate | q.s. |
| Disodium edetate | q.s. |
| Phenoxyethanol | q.s. |
| Fragrance | q.s. |
| Ion exchanged water | balance |
| Total | 100 |

### FORMULATION EXAMPLE 4

### Translucent Whitening Skin Lotion

| | |
|---|---|
| Components added | content(mass %) |

(A) Liposome part

| | |
|---|---|
| Base material of Example 4 | 1 |

(B) Microemulsion part

| | |
|---|---|
| Ethanol | 7 |
| Glycerin | 4 |
| Dipropylene glycol | 5 |
| 1,3-Butylene glycol | 3 |
| POE (10) Methyl glucoside | 1 |
| PEG-20 | 1 |
| PEG/PPG-14/7 Dimethyl ether | 1 |
| Polyoxyethylene (60) hydrogenated castor oil | 0.2 |
| Polyglyceryl-2 diisostearate | 0.1 |
| Isostearyl alcohol | 0.2 |
| Isostearic acid | 0.1 |
| α-Olefin oligomer | 0.1 |
| Dipotassium glycyrrhizinate | 0.1 |
| Tocopherol acetate | 0.1 |
| Tranexamic acid | 2 |
| Scutellaria baicalensis root extract | 0.1 |
| Rehmannia root extract | 0.1 |
| Saxifraga sarmentosa extract | 0.1 |
| Lamium album extract | 0.1 |
| Citric acid | q.s. |
| Sodium citrate | q.s. |
| Trisodium edetate | q.s. |
| Phenoxyethanol | q.s. |
| Fragrance | q.s. |
| Ion exchanged water | balance |
| Total | 100 |

### FORMULATION EXAMPLE 5

### Translucent Whitening Skin Lotion

| | |
|---|---|
| Components added | content (mass %) |

(A) Liposome part

| | |
|---|---|
| Base material of Example 4 | 1 |

(B) Microemulsion part

| | |
|---|---|
| Ethanol | 8 |
| Glycerin | 3 |
| Dipropylene glycol | 5 |
| 1,3-Butylene glycol | 3 |
| POE (10) Methyl glucoside | 1 |
| PEG-20 | 1 |
| PEG/PPG-14/7 Dimethyl ether | 1 |
| Polyoxyethylene (60) hydrogenated castor oil | 0.2 |
| Polyglyceryl-2 diisostearate | 0.1 |
| Isostearyl alcohol | 0.2 |
| Isostearic acid | 0.1 |
| α-Olefin oligomer | 0.1 |
| Dipotassium glycyrrhizinate | 0.1 |
| Ascorbate glucoside | 2 |
| Arginine hydrochloride | 0.1 |
| Potassium hydroxide | 0.4 |
| Scutellaria baicalensis root extract | 0.1 |
| Rehmannia root extract | 0.1 |
| Saxifraga sarmentosa extract | 0.1 |
| Lamium album extract | 0.1 |
| Citric acid | q.s. |
| Sodium citrate | q.s. |
| Disodium edetate | q.s. |
| Paraben | q.s. |
| Fragrance | q.s. |
| Ion exchanged water | balance |
| Total | 100 |

### FORMULATION EXAMPLE 6

### Skin Lotion

| | |
|---|---|
| Components added | content (mass %) |

(A) Liposome part

| | |
|---|---|
| Base material of Example 4 | 1 |

(B) Microemulsion part

| | |
|---|---|
| Polyoxyethylene (60) hydrogenated castor oil | 0.2 |
| Polyglyceryl-2 diisostearate | 0.2 |
| Triethylhexanoin | 0.1 |
| Methyl gluceth-10 | 0.5 |
| 1,3-Butylene glycol | 6 |
| Carboxy vinyl polymer | 0.01 |
| Phenoxyethanol | 0.3 |
| Sodium 3-ethoxysalicylate | 1 |
| Bupleurum falcatum root extract | 0.1 |
| Ethanol | 5 |
| Glycerin | 5 |
| Fragrance | q.s. |
| Ion exchanged water | balance |
| Total | 100 |

### FORMULATION EXAMPLE 7

### Opaque Whitening Skin Lotion

| | |
|---|---|
| Components added | content (mass %) |

(A) Liposome part

| | |
|---|---|
| Base material of Example 4 | 1 |

(B) Microemulsion part

| | |
|---|---|
| Isostearic acid | 0.3 |
| Squalane | 0.1 |
| Cetyl octanoate | 0.1 |
| Behenyl alcohol | 0.1 |
| Stearyl alcohol | 0.05 |
| Polyoxyethylene octyldodecyl ether | 0.1 |
| Sorbitan sesquiisostearate | 0.03 |
| 1,3-Butylene glycol | 0.6 |
| Sodium stearoyl methyltaurin | 0.05 |
| Citric acid | 0.005 |
| Sodium citrate | 0.045 |
| Sodium edetate | 0.01 |
| Inositol | 0.5 |
| Ethyl vitamin C | 1.0 |
| Ion exchanged water | balance |
| Total | 100 |

1. A cosmetic base material, comprising:
(a) a liposome comprising (a-1) lecithin and (a-2) oryzanol, and
(b) an aqueous medium comprising (b-1) water and (b-2) a polyhydric alcohol,
wherein a ratio of a content of said (a) liposome to a content of said (b) aqueous medium ((a):(b)) is within a range from 1:99 to 1:32.

2. The cosmetic base material according to claim 1, wherein said (a) liposome has a mode diameter of 100 nm or less.

3. The cosmetic base material according to claim 1, wherein said (b-2) polyhydric alcohol includes 1,3-butylene glycol and glycerin.

4. A cosmetic preparation comprising (A) a liposome part and (B) a microemulsion part, wherein:
said (A) liposome part consists of the cosmetic base material according to claim 1, and
said (B) microemulsion part is an oil-in-water emulsion comprising (c) an oil, (d) a surfactant, and (e) water.

5. The cosmetic preparation according to claim 4, wherein a content of said (a) liposome is 0.00001 to 0.2 mass % based on the total amount of the cosmetic preparation.

6. The cosmetic preparation according to claim 4, wherein said (d) surfactant comprises (d-1) a hydrophilic surfactant and (d-2) a lipophilic surfactant.

7. The cosmetic preparation according to claim 4, further comprising sodium polyacrylate as (f) a thickener.

8. The cosmetic preparation according to claim 4, further comprising (g) PEG/PPG dialkyl ether.

9. The cosmetic preparation according to claim 4, further comprising (h) a moisturizer.

10. The cosmetic preparation according to claim 9, wherein the (h) moisturizer is diglycerin.
